(19) United States
(12) Patent Application Publication
(10) Pub. No.: US 2014/0165713 A1
(43) Pub. Date: Jun. 19, 2014

(54) SYSTEMS, DEVICES, AND METHODS FOR ENVIRONMENTAL MONITORING IN AGRICULTURE

(71) Applicant: PURESENSE ENVIRONMENTAL INC., Oakland, CA (US)

(72) Inventor: Michelle M. Frey, Oakland, CA (US)

(73) Assignee: PURESENSE ENVIRONMENTAL INC., Oakland, CA (US)

(21) Appl. No.: 14/017,182

(22) Filed: Sep. 3, 2013

Related U.S. Application Data
(63) Continuation of application No. PCT/US12/27588, filed on Mar. 2, 2012.
(60) Provisional application No. 61/473,002, filed on Apr. 7, 2011.
(60) Provisional application No. 61/449,547, filed on Mar. 4, 2011.
(60) Provisional application No. 61/449,533, filed on Mar. 4, 2011.

(51) Int. Cl. G01N 33/24

(52) U.S. Cl. CPC G01N 33/24
USPC 73/64.56; 73/864

(57) ABSTRACT

Embodiments of the present disclosure relate generally to nutrient monitoring in an agricultural field, and more specifically to devices, systems and methods that provide real time analysis and monitoring of one or more nutrients using ion selective electrodes.

### CROSS-REFERENCE TO RELATED APPLICATIONS

This application is a continuation of PCT Application No. PCT/US2012/027588, filed on Mar. 2, 2012, which claims the benefit of U.S. Provisional Application No. 61/473,002, filed on Apr. 7, 2011, U.S. Provisional Application No. 61/449,547, filed on Mar. 4, 2011, and U.S. Provisional Application No. 61/449,533, filed on Mar. 4, 2011, each of which is incorporated by reference herein in its entirety.

### TECHNICAL FIELD

Embodiments of the present disclosure relate generally to systems, devices, and methods for the monitoring of nutrients in the environment. In some embodiments, the systems, devices, and methods are adapted for use in agricultural settings; however, the systems, devices, and methods may be adapted for use in non-agricultural settings including residential, commercial and experimental settings.

### BACKGROUND

The agriculture industry employs wide use of nitrogen-based fertilizers to increase crop production. Assuring the availability of fertilizer compounds is critical for growers in achieving desired yield and quality of crop production. To meet these objectives, growers apply fertilizers at levels much greater than what their crops can utilize. Generally, nitrogen applied to an agricultural field will be either dissolved or will enter the soil environment in its dissolved forms, such as nitrate (NO³⁻), nitrite (NO²⁻), ammonium (NH⁴⁺), and urea ((NH₂)₂CO). Common forms of nitrogen-based fertilizers include Urea Ammonium Nitrate 32% (UAN-32) and Calcium Ammonium Nitrate 17% (CAN 17).

The availability of nitrogen to the crop is often limited and significant losses from the soil profile can occur. For example, the availability of nitrogen to the crop or plant may be limited by a number of mechanisms such as: nitrogen remaining adsorbed onto the soil particles as opposed to being absorbed by the roots of the crop, nitrogen migrating through the soil pore water to soil depths or lateral areas that are away from the crop's root system and, in the absence of oxygen, nitrogen can be reduced to free nitrogen gas and released to the atmosphere.

Sorption onto soil particles is negligible for nitrate and nitrite species of nitrogen, and only weak for ammonium. Urea, however, has a stronger sorption potential and can be retained by soils and therefore, effectively removed from the soil pore water environment. Over time, oxidation of sorbed nitrogen compounds can occur during future irrigation events, resulting in the more soluble forms of nitrogen (nitrate and nitrite) being released into the soil pore water environment.

Nitrification in soil environments is necessary to transform organic nitrogen or soil-bound sources of nitrogen into inorganic forms readily mobilized for uptake by plants. Facultative bacteria in the soil environment breakdown organic nitrogen into ammonium and then subsequently oxidize the ammonium into nitrite and then nitrate. The key environmental conditions necessary to initiate and complete the nitrification processes include: active microbiological environment with nitrifying bacteria; available oxygen in soil pore environment, either as dissolved oxygen in the soil pore water or free available oxygen that can be scavenged from the gaseous phase; organic nitrogen or ammonium in contact with the nitrifying bacteria; and soil pore water having a pH between 7.0 and 8.5 (Sahrawat, K. L., 2008; Factors Affecting Nitrification in Soils, Communication in Soil Science and Plant Analysis, 39 (9-10): 1436-1446; van Haandel, A. et al., J., 2007; Handbook Biological Waste Water Treatment Design and Optimisation of Activated Sludge Systems, Quist Publishing (Leidschendam, The Netherlands), 570 pp.; Montagnini F. et al, 1989, Factors Controlling Notrifications in Soils of Early Successional and Oak/Hickory Forests in the Southern Appalachians, Forest Ecology and Management, 26: 77-94; Patrick, W. H. et al., 1976, Nitrification-Denitrification Reactions in Flooded Soils and Water Bottoms: Dependence on Oxygen Supply and Ammonium Diffusion, Journal of Environmental Quality, 4: 469-472). While temperature is also an important factor to the rate of nitrification, the temperatures found during crop production cycles are high, so its contribution is negligible.

Therefore, to anticipate the fate and transport of nitrogen in the soil environment, monitoring for pH and dissolved oxygen can provide important indicators of the likelihood that nitrification processes are enabled or inhibited in the background soil-water-air environment.

Once nitrogen is present in the forms of either nitrate (NO³⁻) or nitrite (NO²⁻), the retention of these compounds in the soil-water-air environment is very poor. If these constituents are not consumed by plant uptake, the remaining concentrations are available to migrate beyond the effective root zone and therefore contribute to a potential environmental risk. The migration of nitrate/nitrite to underlying aquifer systems is a known and serious environmental concern. While vertical migration of these compounds to deep strata aquifers may take decades or beyond, the “business” of agriculture has an equally long life. Conditions of potable water supplies being affected by nitrate migration off-farm in California are being experienced. Judicious management of nitrogen use is critical to the long-term environmental sustainability of agriculture as the reclamation of water supplies for nitrate/nitrite removal is both difficult and extremely costly.

To maximize crop production, growers need to make on-going decisions about the nutrition needs of their crops. Current practices use representative soil samples collected mutually from a grower's field to determine the background nutrient content that could be readily extracted by the crop. These samples are analyzed using standard laboratory procedures for nutrient extraction and analysis as exemplified in the North Central Region (NCR-13) Committee, Recommended Chemical Soil Test Procedures for the North Central Region, North Central Regional Research Publication No. 221(1998), Missouri Agricultural Experiment Station SB 1001: 75 pp. Using published guidelines on nutrient requirements for a given crop, a grower then applies nutrients at an appropriate rate over the course of the crop's growth cycle.

Growers also need to be able to manage the nutrient content of their soils to optimize nutrient applications for crop production while balancing the potential for environmental degradation from excess nutrient applications. Today's practices require growers to take soil samples through the growing season and use laboratory methods to assess the nutrient content available in their soils. This information, while valuable, does not provide growers with information about the true availability of nutrients over time for crop uptake; nor does it assist the grower in assessing the correct usage of nutrients by the crop as opposed to losses to soil horizons below the crop's root zone. It is valuable for growers to be able to track throughout the growing cycle the changing character of nutrients and their availability to the crops being produced. Among other things, the existing practices and technologies available today do not provide for the automated obtainment and/or analysis of soil pore water, do not provide the capability to take repeated soil pore water measurements at periodic intervals without manual intervention, and do not allow real-time trends in specific nutrient content in soil environments to be identified over time in such a way that maintains long-term sensor performance stability and accuracy. New developments and improvements are needed.

### BRIEF SUMMARY

The systems, devices, and methods described herein can be installed in a grower's field to monitor soil pore water quality conditions. To track the available nutrients in the soil profile, measurement of the soil pore water is critical. Soil pore water is broadly defined as water that is found to occupy the void spaces between and around soil particles. Of particular interest is the nutrient content of soil pore water located in the root zone of a grower's crops. The systems, devices, and methods described herein provide information and data about trending nutrient behavior in the soil environment over time. Growers can then assess the total available nitrogen lost from either root uptake or migration out of the root zone, and the grower can identify triggers for the addition of nutrients or flags about excessive nutrient content that could be harmful either to the crop or as an environmental concern. The present subject matter pertains to the delivery of real-time data about soil pore water nutrient content, as well as pragmatic and useful tools for growers to improve their nutrient management programs.

In some example embodiments, a device is provided comprising: a sample collection unit configured to collect soil pore water samples at one or more depths in a soil environment and a measurement unit coupled to the sample collection unit and configured to analyze the soil pore water samples to determine the level of at least either one nutrient or key environmental parameter important to estimating the fate of nutrients in the soil pore water samples.

The sample collection unit may comprise an elongate tube having one or more collection chambers formed therein, where the one or more collection chambers are located at varying depths along the length of the tube. Each collection chamber generally includes one or more openings in the chamber wall to allow soil pore water to flow in.

In an illustrative embodiment, the sample collection unit includes a sampling assembly. The sampling assembly may comprise a manifold; one or more micropumps coupled to the manifold; a sampling reservoir; and a plurality of sampling lines. Generally, each of the sampling lines is independently coupled to the manifold and has an open distal end. This open distal end of at least one of said sampling lines extends into a collection chamber to draw a soil pore water sample therefrom.

To detect one or more nutrients in the samples, the measurement unit includes one or more ion selective electrode (ISE) sensors. The ISE sensors are configured to receive the soil pore water samples and detect ions or anions representative of the nutrient content and/or environmental quality parameters of each of the soil pore water samples. Data representative of at least one characteristic of the nutrients is analyzed by a microprocessor in the measurement unit. The microprocessor is configured to transform the measurement result into digitized engineering units, store the results, parse the results into a defined organization of data elements, and manage the transmission of data results via user download from the device or wireless transmission using a data results via user download from the device or wireless data servers remote from the agricultural field.

In another embodiment, a system for monitoring or analyzing nutrients in an agricultural field is provided comprising: a sample collection unit configured to collect one or more samples at one or more locations in the agricultural field; a measurement unit, coupled to the sample collection unit, and configured to monitor or analyze the samples to determine the contest of at least one nutrient in the samples; and a data acquisition and communications unit, coupled to the measurement unit, and configured to transmit nutrient data to one or more data servers remote from the agricultural field.

In yet other embodiments, methods of monitoring nutrients are provided, comprising the steps of: collecting one or more samples in an agricultural field, said samples containing one or more nutrients; detecting at least one of the nutrients in the sample and generating data representative of at least one characteristic of the nutrient; and transmitting the data to one or more data servers remote from the agricultural field. In some embodiments, ions or anions of interest in the sample are detected using at least one ion selective electrode (ISE) sensor.

In further embodiments, methods of monitoring nutrients in an agricultural field are provided characterized in that: one or more samples in the field are collected, nutrient content in the one or more samples is detected and data is generated representative of the nutrient content or other characteristics, all of the foregoing steps occurring in the field, and wherein the data is transmitted to a data server remote from the field. In some embodiments, the one or more samples comprise soil pore water collected at varying depths in the field. Alternatively, or additionally, the one more samples may comprise irrigation water collected at the field. The one or more samples may also comprise portions of crop canopy or crop fruit collected at the field. A non-exhaustive list of examples of nutrients, the content of which can be determined, include at least nitrogen, phosphorous, potassium, and boron.

An environmental quality parameter is referred to herein as a parameter, metric, or characteristic of soil pore water that indicates the quality of the growing environment for a particular crop. A non-exhaustive list of examples of environmental quality parameters that can be determined include at least calcium content, magnesium content, carbonate content, sulfur compound content, pH, electroconductivity, and temperature.

Of particular advantage, nutrient measurement and/or monitoring at multiple depths in the soil environment, and optionally measurements and/or monitoring of irrigation water and the crop canopy itself, all using a single stand-alone device or system, are provided herein.

In some embodiments, remote calibration and remote diagnosis of devices, such as nutrient monitoring devices installed in agricultural settings, for example in a grower's field used to monitor soil pore water quality conditions, are provided.

In some embodiments, methods for remotely diagnosing selected operational parameters of a device are provided. Optionally, automated notifications regarding maintenance and/or service needs are generated at the device and communicated to remote servers.

In some embodiments, a method for remotely diagnosing ISE sensor performance is provided. ISE sensors may be used in nutrient monitoring devices, such as the systems and devices described in co-pending U.S. Provisional Patent Application No. 61/449,533 (attorney docket no. 052579-007) entitled “In-situ Sampling and Monitoring Device for Real-time Nutrient Monitoring in Agricultural Fields,” and filed Mar. 4, 2011, the entire disclosure of which is herein incorporated by reference. Of particular advantage, embodiments of the method diagnose ISE sensor drift by monitoring sensor drift remotely and generating maintenance and/or service notifications when certain sensor values move or fall outside of set or user defined values and limits. Automated data adjustment can be made to compensate for such drift. Methods for the automated data adjustment to compensate for drift are defined to include multiple algorithms that are based on field device measurement operation for comparing measurements that have been identified as representing comparable conditions. Drift is the offset between these comparable set of field measurements. Routine measures can be automatically adjusted based on the resultant offsets found by the algorithms used to determine sensor measurement drift.

Systems, devices, and methods are also provided to allow proper operation and performance of ISE sensors is maintained by delivering multiple fluids needed to sustain, clean, and calibrate the deployed ISE sensors.

In some embodiments, operational health of a nutrient monitoring device is monitored and transmitted from the device to a data acquisition system associated with the device. Parameters representative of the operational health of the device may be user defined. Parameters representative of the operational health of the device may include, but are not limited to, measures and assessments such as any one or more of: power supply, sensor responsiveness, sensor drift, sensor outlier values, vacuum pump energy, vacuum pump operational status, vacuum pump hours from last maintenance and total hours, deionized (DI) water reservoir volume, and DI water reservoir refill flags.

In some embodiments, the nutrient monitoring device is equipped with multiple solute reservoirs configured to deliver calibration and sensor tip cleaning solutions, such as for example when ISE sensors are used. These sensor tip cleaning solutions may be provided in addition to DI water. These solutions are used when diagnosis of the ISE sensor health shows that a sensor is operating outside of its performance specifications as established by the user. Tip cleaning and recalibration may be performed in an automated manner controlled by a microprocessor and associated firmware.

Also provided herein are methods of diagnosing sensor performance in a nutrient monitoring device installed in an agricultural field, the device having a sample reservoir and one or more sensors for detecting the nutrient content of a sample. In one example, when the device is not in operation, a solute is circulated in and through the sample reservoir and the one or more sensors. Sensor signals are acquired at predefined intervals. Data from the sensor signals is transmitted to a data server remote from the device, and this data front the sensor signals is compared to predefined values to diagnosis sensor performance. The solute may be any suitable solution and will vary depending of the type of device and the type of sensors used in the device. In some embodiments, the solute is a calibration standard. In other embodiments, the solute is DI water. Further, multiple solutes may be employed to represent different solution concentration standards, cleaning solvents for ISE maintenance, and DI water.

The methods described herein may be employed with a variety of different nutrient monitoring devices. Any suitable sensor may be used. In some embodiments, the one or more sensors are Ion Selective Electrode (ISE) sensors.

Of particular advantage, some embodiments provide for calibrating the nutrient monitoring device based on the comparison of data from the sensor when producing measurements for a calibration standard solution of known concentration or value. The remote monitoring device would be operated by firmware instructing the device to operate in “Calibrate” mode where the following steps are performed: (1) sample lines to the sensor are flushed with DI water for a designated period of time; (2) standard solutions are circulated in the sampling line for the sensor while readings are captured during a designated time period; (3) standard solutions are sequenced in their circulation from the lowest concentration or value to the highest concentration or value; (4) DI water is circulated in the sampling line for a designated period of time; and (5) the remote monitoring device is returned to routine operation.

Of further advantage, cleaning of the sensors in the nutrient monitoring device is remotely initiated. For example in one embodiment one or more cleaning solutions are circulated through the one or more sensors. This cleaning step may be initiated on a user defined schedule. Alternatively and/or optionally the cleaning step is initiated when the comparison of data from the sensor signals to predefined values is outside of predefined limits.

Other systems, devices, methods, features and advantages of the subject matter described herein will be or will become apparent to one with skill in the art upon examination of the figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The accompanying drawings, which are incorporated into this specification, illustrate one or more exemplary embodiments of the inventive subject matter disclosed herein and, together with the detailed description, serve to facilitate explanation of some of the principles and exemplary implementations of the inventive subject matter. One of skill in the art will understand that the drawings are illustrative only, and that what is depicted therein may be adapted based on the text of the specification and the spirit and scope of the teachings herein.

In the drawings, where like reference numerals refer to like reference in the specification:

FIG. 1 is a schematic and cross-sectional view of an example embodiment of a nutrient sampling and monitoring device;

FIG. 2 is a block diagram of an example embodiment of a nutrient sampling and monitoring device;

FIG. 3 is a depiction of an example embodiment of a nutrient sampling and monitoring system deployed in an agricultural setting;

FIG. 4 is an in-field layout of an example embodiment of a measurement unit deployed without a soil pore water sample collector;

FIG. 5 is a chart showing sample data from real-time in situ monitoring of soil moisture and electroconductivity (Ec) in ambient soil environments (hourly averages of 15-minute increment data displayed);

FIG. 6 is a flowchart depicting an example embodiment of a nutrient monitoring method;

FIG. 7 is a flowchart depicting an example embodiment of a nutrient sampling method;

FIG. 8 is a graph illustrating an example of sensor “drift;”

FIG. 9A-C are illustrations of examples of various corrections for sensor “drift;”

FIG. 10 is a flow chart illustrating an example embodiment of a deionized water sampling method;

FIG. 11 is a flow chart illustrating an example embodiment of a sensor cleaning method; and

FIG. 12 is a flow chart illustrating an example embodiment of a sensor calibration method.

### DETAILED DESCRIPTION

Various example embodiments are described herein in the context of the monitoring of nutrients which is particularly suitable for, but not necessarily limited to, agricultural settings.

Those of ordinary skill in the art will understand that the following detailed description is illustrative only and is not intended to be in any way limiting. Other embodiments may likely suggest themselves to those persons of ordinary skill in the art having the benefit of this disclosure and the teachings provided herein.

In the interest of clarity, not all of the routine features of the example embodiments described herein are shown and described. It will of course be appreciated that in the development of any such actual implementation, numerous implementation-specific decisions must be made in order to achieve the specific goals of the developer, such as compliance with regulatory, safety, social, environmental, health, and business-related constraints, and that these specific goals will vary from one implementation to another and from one developer to another.

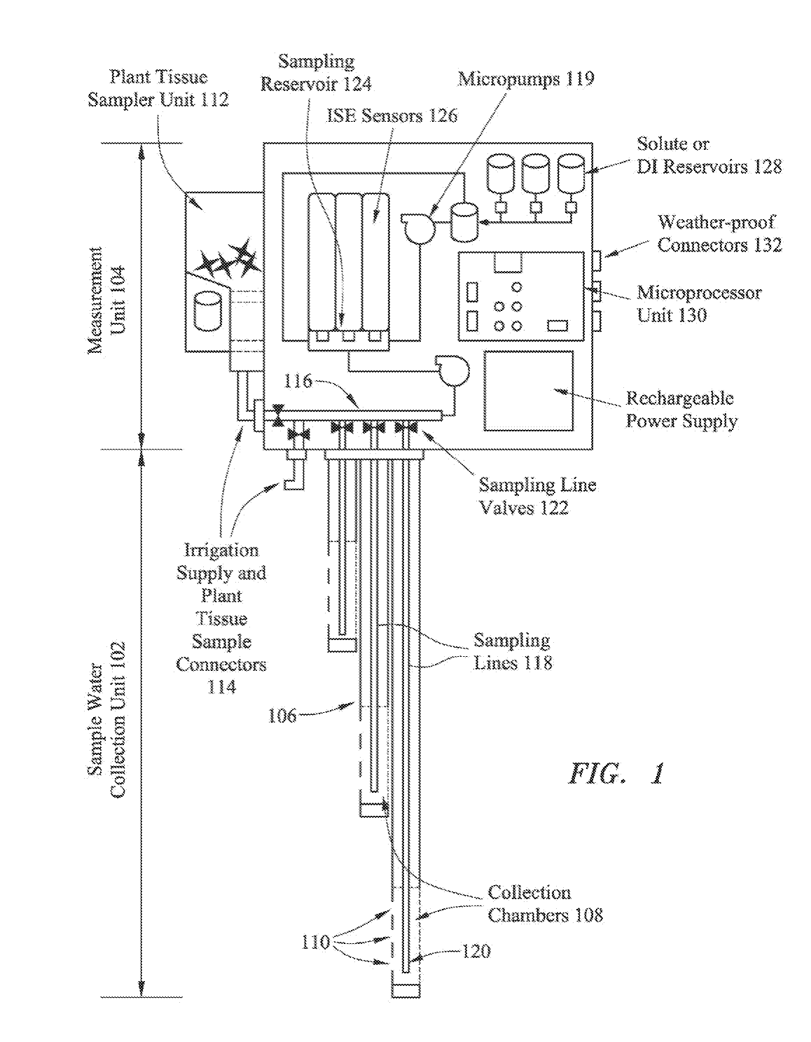

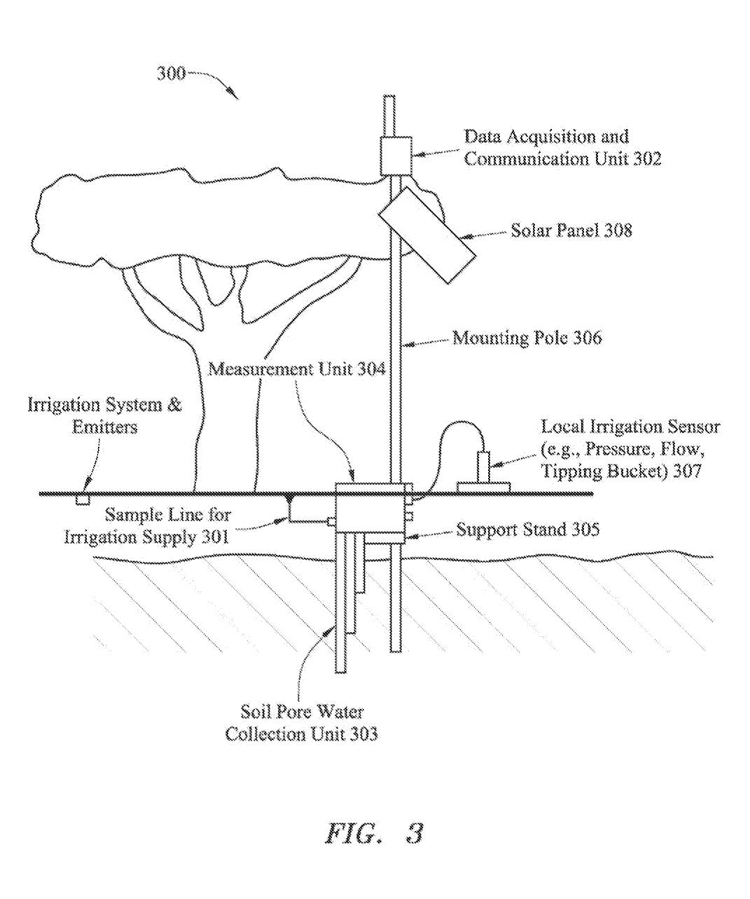

In general systems, devices, and methods are provided that can be installed in a grower's field to monitor soil pore water quality conditions and transmit that information front the field to one or more remote data servers. A field monitoring device is described that can provide liquid samples from soil, the irrigation system, and plant tissue for analysis using commercially available Ion Selective Electrode (referred to herein as ISE or ISEs) sensor(s). Data from the field sampling and measurement device is further processed, managed, stored, and transmitted to remote data servers using the devices and systems described herein and, e.g., illustrated by FIG. 1 and FIG. 3. The field sampling and measurement device can be deployed in the field for a matter of months or over a year.

Monitoring the chemical properties of in-situ soils using ion selective electrodes (ISE) deployed for long periods of time is made difficult today by the environmental factors that affect ISE measurement performance over time. Many of the embodiments described herein allow such deployments to be delivered for field monitoring of soil pore water quality under wetting conditions such as irrigation or rainfall in the zone of greatest root activity. Within a bored hole, the collection device is inserted where multiple chambers are housed to collect soil pore water along the depth of the soil profile. The depth will vary depending on the type of agricultural use and by the crop type.

Also described are systems and methods that provide remote calibration and diagnosis of devices, such as nutrient monitoring devices installed in agricultural settings, such as in a grower's field used to monitor soil pore water quality conditions.

Embodiments of methods for remotely diagnosing selected operational parameters of a device are also provided. Optionally, automated notifications regarding maintenance and/or service need are generated at the device and communicated to remote servers.

In other embodiments, methods for monitoring the operational health of a device and transmitting data from the device to a data acquisition system associated with the device are described. Parameters representative of the operational health of the device may be user defined. Parameters representative of the operational health of the device may include, but are not limited to, measures and assessments such as any one or more of: power supply, sensor responsiveness, sensor drift, sensor outlier values, vacuum pump energy, vacuum pump operational status, vacuum pump hours from last maintenance and total hours, DI water reservoir volume, and DI water reservoir refill flags.

In some embodiments, the nutrient monitoring device is equipped with multiple solute reservoirs configured to deliver calibration and sensor tip cleaning solutions, such as for example when ISE sensors are used. These sensor tip cleaning solutions may be provided in addition to DI water. These solutions are used when diagnosis of the ISE sensor health shows that a sensor is operating outside of its performance specifications as established by the user. Tip cleaning and recalibration may be performed in an automated manner controlled by a microprocessor and associated firmware.

In general, the field sampling device 100 is comprised of a sample collection unit 102 and a measurement unit 104 as illustrated in FIG. 1. The sample collection unit 102 and the measurement unit 104 may be comprised of one physically integral unit, or the measurement unit 104 can be located without a sample water collection unit 102. When co-located, the sample water collection unit 102 is connected to the measurement unit 104 by a “snap on” connector that aligns the mechanical components so that soil pore water samples can be extracted from the sample water collection unit 102.

The sample collection unit 102 for soil pore water sample collection is generally comprised of an elongate assembly of one or more sample collection tubes 106 that each include a collection chamber 108. The assembly can support multiple sample collection tubes 106 of various lengths so that samples can be collected at various depths within the soil profile. In some embodiments the assembly 106 is preferably made of a rigid material for durability; however, other materials may also be used. The unit 102 is installed in the field. Generally, an augered hole of the same or similar size as the diameter of the assembly of tubes 106 is created or bored into the soil environment in an agricultural field or other desired location. The assembly of tubes 106 is then placed into the hole as shown in FIG. 3.

The depth of roots produced in the soil environment depends on the crop type, and therefore, the relevant depths used for a particular installation of the sample water collection unit also depend on the crop. Multiple tubes are included in the assembly 106 in order to profile the migration of nutrients from the more shallow to the complete depth of the root zone. In one embodiment, the depth of the hole for monitoring will be within the top 36 inches so as to focus the information on the predominant area of the crop root zone. Typically, in some embodiments, the range of sample depth intervals is 6 to 12 inches with a total assembly of tubes ranging from three to five units for a single sample water collection unit. Thus, the total depth of the sample water collection unit can, e.g., range from as low as 6 inches to as deep as 60 inches.

The walls of the collection chambers 108 include one or more holes, openings or vents 110 along the length and circumference of the sample collection unit 102 at spaced intervals to allow soil pore water to flow into the collection chambers 108 located at the same depth. The walls of the collection chambers 108 may also be (or alternatively be) porous. Typically the openings are configured such that water may flow into the collection chambers 108, while soil, rock and other solid material does not pass through. Water seeps into the collection chambers 108 during wetting events. For purposes of this description a wetting event is defined as irrigation, rainfall, or any event or environmental condition that provides sufficient soil moisture to be retained for extraction. Soil moisture is considered sufficient when there is enough moisture extracted from the soil by the sample collection unit to be analyzed by the measurement unit. For example, in some embodiments, it is observed that about 5 milliliters (mL) of water is necessary for measurement.

The sample collection unit 102 is communicatively coupled to the measurement unit 104. In one example, the sample collection unit 102 is connected to the measurement unit 104 by miniaturized “snap-on” connectors so that the sampling lines are aligned property to have soil pore water samples flow directly into the collection manifold 116. The sample collection unit 102 generally integrates the flow of one or more samples to ISE sensor(s) from either an external water source (such as the irrigation supply), the soil pore water, or, with an attachment to the unit, samples derived from a plant tissue processing unit 112. Samples obtained from the irrigation supply enter the collection manifold 116 via an irrigation supply port 114.

To manage the flow of samples, a sampling assembly is provided that includes a manifold 116 and a plurality of sampling lines 118. One or more micropumps 119 are coupled to the manifold and sampling lines. Generally, each of the sampling lines 118 is independently coupled to the manifold 116 and has an open distal end 120. This open distal end 120 of at least one of said sampling lines 118 extends into each of the collection chambers 108 to draw soil pore water samples from each of the collection chambers up through the manifold 116 via valves 122 and into a sampling reservoir 124. Preferably, there is one sampling line 118 per chamber 108. Once samples have been drawn up into the sampling reservoir 124, detection of one or more nutrients in the samples using one or more ISE sensors housed in an ISE sensor chamber 126 may begin. The samples can be drawn concurrently into separate reservoirs each having its own ISE sensor, or concurrently into a common reservoir with one ISE sensor to obtain a blended measurement. The samples can also be drawn sequentially, i.e., one after the other.

The measurement unit 104 can house the ISE sensor chamber 126, micropumps 119, all electronics, battery power supply, and reservoirs of various fluids as needed for analyses, including DI water (described in more detail below). However, older configurations are possible and within the scope of this disclosure.

Monitoring the chemical properties of in-situ soils using ISE sensors deployed for long periods of time is made difficult today by the environmental factors that affect ISE unit measurement performance over time. However, these difficulties are reduced and/or eliminated by the subject matter disclosed herein, which provides for ISE sensor deployments for field monitoring of soil pore water quality under wetting conditions in the zone of greatest root activity.

When the ISE sensors are not in a measurement state, a continuous supply of DI water from DI reservoir 128 is re-circulated through the ISE sensor chamber 126 to maintain a wetted environment for the ISE sensors, when needed.

Of particular advantage, the field sampling device 100 includes a microprocessor unit 130 configured to manage the power supply delivery, operation of the micropumps, sensor data recordings and data transmission to data acquisition systems using standard communication protocols, among other functions. Valve controls are managed by the measurement unit microprocessor in terms of sample collection frequency and clean sample flushing with DI water between samples, as needed.

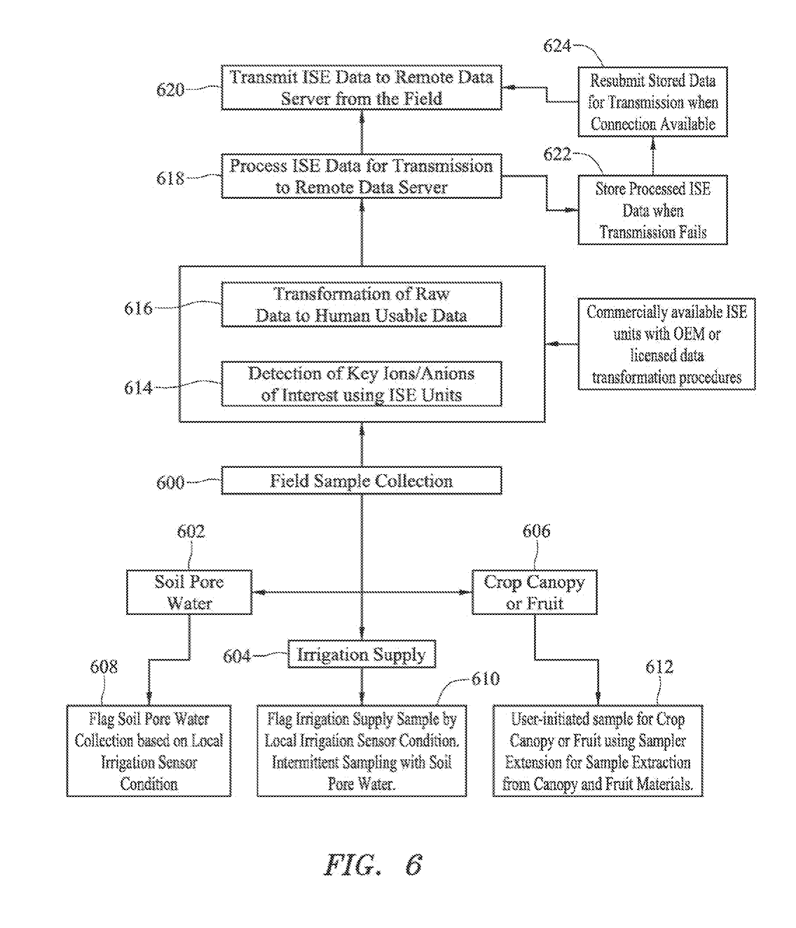

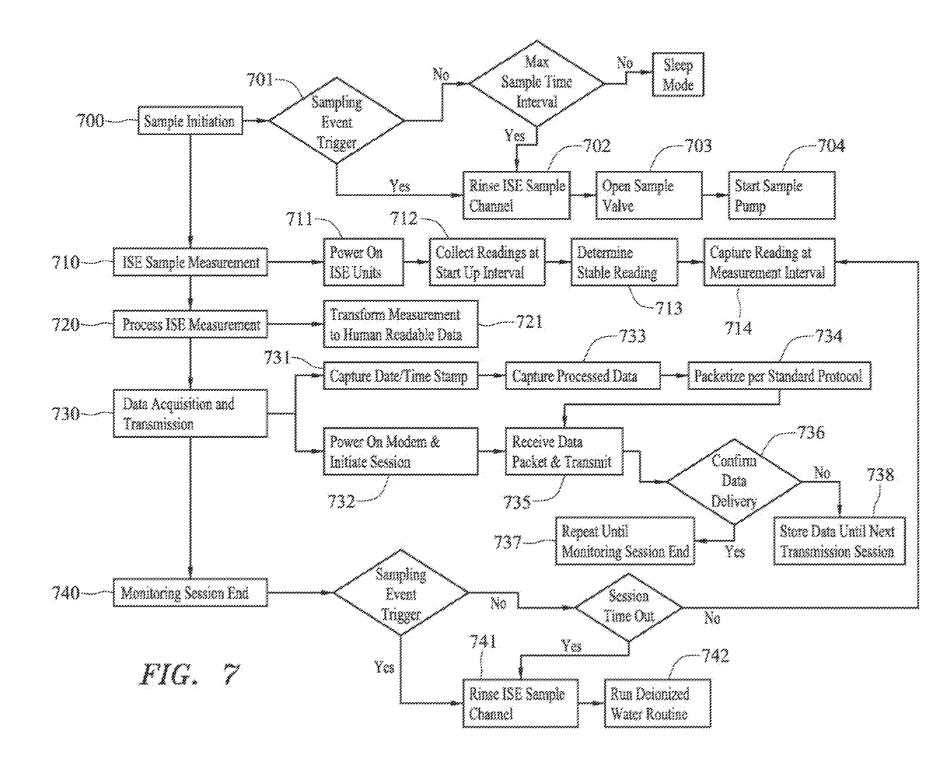

The microprocessor 130 may include weather-proofed connectors 132 coupled thereto to enable additional functions such as solar panel recharge of the power supply, connection to the data acquisition and transmission unit, and the receiving of in-coming signals from additional sensors that may be useful for the operational logic of the measurement unit 104. Operation of the microprocessor is described in more detail below with reference to FIGS. 6-7.

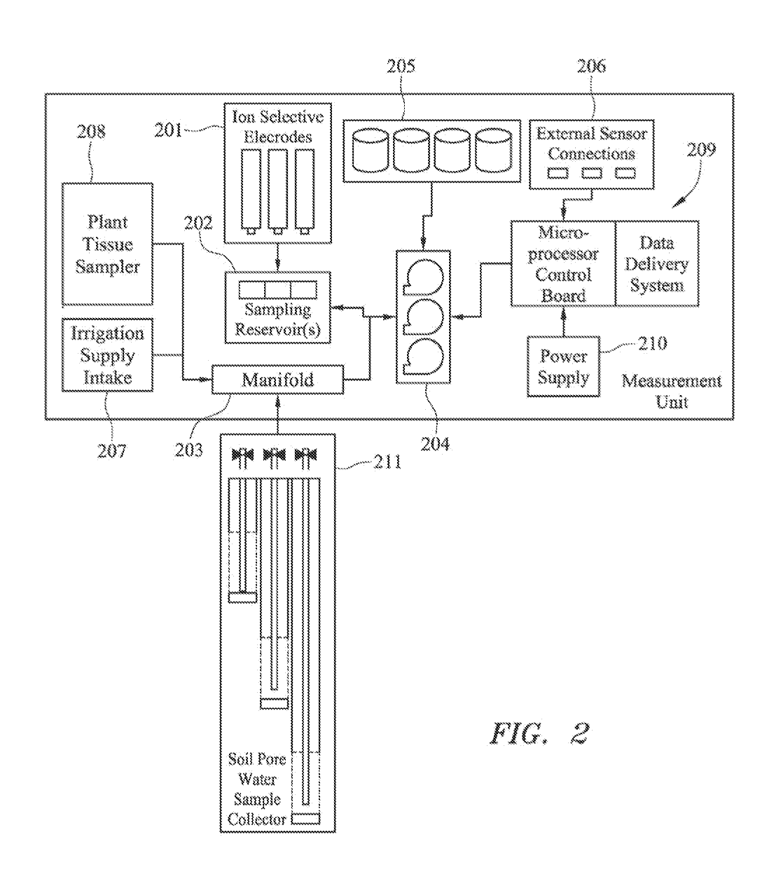

The measurement unit and sample water collection unit are separable to allow use of the measurement unit alone without the sample water collection unit, and to allow rapid exchange of one unit for another should an upgrade, revision, or replacement be needed. Referring to FIG. 2, a functional layout of a monitoring and sampling device is provided. For example, the device may comprise a soil pore water sample collector 211 operatively connected to a measurement unit 200. The soil pore water sample collector 211 may be integrated with or physically separated from the measurement unit 200.

The measurement unit 200 may comprise one or more of the following components: a plant tissue sampler 208, an irrigation supply intake 207, a manifold 203, one or more sampling reservoirs 202, one or more pumps 204, a power supply 210, a microprocessor control board and data delivery system 209, one or more external sensor connections 206, one or more solute or DI reservoirs 205 and one or more ion selective electrodes 201. In the illustrative embodiment shown in FIG. 2, the above-referenced components are provided inside a single measurement unit 200; however, unit 200 is not limited to this configuration and one or more of the components may be housed separately, and/or located physically remote from the measurement unit 200.

In the illustrative embodiment shown in FIG. 2, the plant tissue sampler 208 and the irrigation supply intake 207 are operatively connected to the manifold 203. The manifold 203 is operatively connected to the one or more sampling reservoirs 202 and the one or more pumps 204. The one or more ion selective electrodes (ISEs) 201 are operatively connected to the one or more sampling reservoirs 202 (in one embodiment having plural reservoirs, the connections may be made in parallel). The one or more solute or DI reservoirs 205 are operatively connected to the one or more pumps 204. The external sensor connections 206 and the power supply 210 are operatively connected to the microprocessor control board and data delivery system 209, and the microprocessor control board and data delivery system 209 are operatively connected to the one or more pumps 204. Each of the above-referenced components may be modular to facilitate maintenance, replacement, enhancement and the like.

This modularization also enables rapid replacement and easy maintenance and support in the field. Each modular component represents a function within either the measurement or sample water collection units.

At the core of the sampling device is the arrangement of ion selective electrodes (ISEs) which according to some embodiments are delivered to the measurement unit as cartridges 201 that can be easily inserted and/or removed. The ISEs may be inserted into a continuous sampling reservoir unit 202 or may have a dedicated sampling reservoir that can be easily connected to other ISE reservoir units.

Samples enter the measurement unit 200 through the sample collection manifold 203, and the flow of samples are driven to the sampling reservoir using the micropump assembly 204. When the measurement unit 200 is not actively performing sample analyses, the micropumps 204 are used to move various fluids or solutions, such as deionized water (default routine operation) or special solutions used for sensor cleaning and calibration from the reservoir assembly 205.

Operation of the measurement unit 200 in terms of the micropump operation and solution movement through the sampling reservoir 202 is triggered based on set points for measurements collected from external sensors 206 connected to the measurement unit such as soil moisture, irrigation system pressure, or rainfall.

The measurement unit 200 can receive samples from three different collection systems: namely from (1) soil pore water sample collector 211, (2) irrigation water supply intake 207, and (3) plant tissue sampler 208.

In some embodiments, the soil pore water sample collector 211 is comprised of one or more sample collection tubes installed into the soil profile. Any number of sample collection tubes may employed. In the illustrative embodiment, one to five sample collection tubes are used.

The irrigation water supply can enter the manifold through the irrigation water supply intake 207 using sample line tubing connected to the irrigation supply line available at the location of the measurement unit 200.

The plant tissue extraction sampler 208 enables plant tissue to be analyzed. In some embodiments, the plant tissue extraction sampler 208 is configured to grind the plant tissue and to expose the ground plant tissue to extraction solvents which solubilize nutrient ions present in the plant tissue. The resulting plant tissue solution is then delivered to the sampling reservoir 202. Those of ordinary skill in the art will readily recognize the structure of plant tissue sampler 208.

Control of the operation of the measurement unit, including all mechanical and electrical systems, is performed by a microprocessor control board 209 where resident software is used to deliver the control logic for operation of the overall system. The microprocessor control board 209 further comprises a data delivery system with one or more modems which can be provided within the mechanical enclosure of the measurement unit or in a separate mechanical enclosure (for example, NEMA-4) for mounting above crop canopy when necessary. Power is supplied to the measurement unit 200 and all related components, including external sensors, using a rechargeable power supply 210 where solar panels can be used to recharge the power supply.

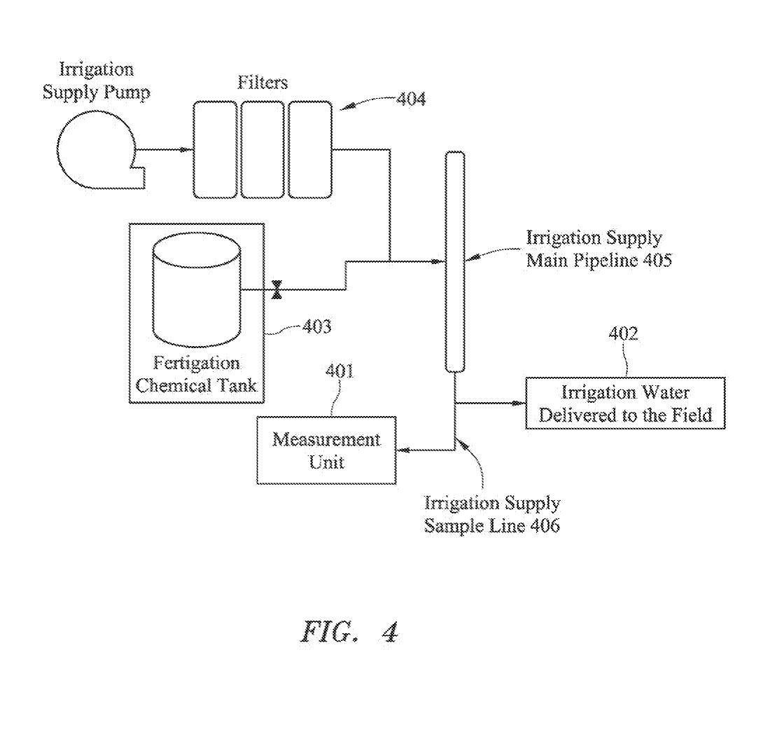

As described above, the systems are modular, and this modularity of both the measurement unit 104 and the sample water collection unit 102 supports delivery of the system in at least two field installation configurations, for example where the measurement and sample collection units are combined and deployed in the field as shown in FIG. 3, and where the measurement unit is deployed in the field without the sample collection unit as shown in FIG. 4.

Referring in detail to FIG. 3, a field installation system 300 is shown. Of particular advantage, system 300 provides a fully integrated, stand-alone system capable of providing real-time nutrient monitoring and data transmission direct from the field. The system 300 broadly comprises a sample collection unit 303 (which may be similar to sample collection unit 102), a measurement unit 304 (which may be similar to measurement unit 104), and data acquisition and communications unit 302. The data acquisition and communications unit 302 is coupled to the measurement unit 304, and is configured to transmit nutrient data to one or more data servers (not shown) located remote from the agricultural field.

System 300 preferably, but not necessarily, further comprises the following features: structural support and mounting systems such as support stand 305 and mounting pole 306 and the like; local irrigation sensors 307; and local power supply management using solar panels 308 that provide recharging of available battery power units. A microprocessor unit housed in the measurement unit 304 is configured to manage data acquisition from the measurement unit 304, data storage and data transmission to wireless telemetry units (not shown) which ultimately deliver the data to internet-hosted servers, or optionally to deliver data to one or more wireless LAN and WAN networks. Optionally, system 300 may further comprise an irrigation system and emitters, which may be connected to the measurement unit 304 via a sample line for irrigation supply 301 or the like. As referred to above, several embodiments are directed to methods of monitoring or analyzing nutrients in an agricultural field.

In this illustrative embodiment, the integrated measurement and sample collection units are delivered when soil pore water samples are to he collected in order to represent nutrients and environmental conditions in crop production environments. In this case, the sample water collection unit 303 is configured to meet the specific requirements of the given crop in terms of the overall depth of the effective root zone of the crop and the sampling depth interval desired. Any arrangement of sample collection tubes may be used, and typically up to five separate depths of sample collection tubes 303 can be configured to meet site specific requirements. The installation of the system in the field mounts the measurement unit 304 onto a structure that can support a solar panel 308, data acquisition and communication unit delivered separately from the measurement unit 302, mounting pole 306, and support stand 305. Sample lines from the irrigation supply line deliver irrigation water to the measurement unit sample collection manifold 301. The assembly of soil pore wafer sample collection tubes 303 is installed into the soil profile. At least one external sensor 307, such as a pressure sensor, provides data to the measurement unit that can be used to trigger sampling event sequences.

FIG. 4 illustrates an alternative embodiment and shows a schematic in-field layout of a measurement unit deployment without a soil pore water sample collection unit. For example, the device may comprise an irrigation supply pump 407 operatively connected to one or more filters 404, which are operatively connected to an irrigation supply main pipeline 405. Also operatively connected to the irrigation supply main pipeline 405 is a fertigation system or station 403 generally comprised of one or more fertigation chemical tank(s) and associated piping. A valve may be provided between the fertigation chemical tank and the irrigation supply main pipeline 405 to control application of fertilizer to irrigation water 402 delivered to the field. The irrigation supply main pipeline 405 is operatively connected to a measurement unit 401 via an irrigation supply sample line 406. The irrigation supply main pipeline 405 supplies irrigation water 402 to be delivered to the field.

A similar physical assembly of mounting pole, solar panel, and support stand are used when delivering the measurement unit alone 401 without a sample water collection unit. Sample types that are processed by the device when configured as a measurement unit alone are typically the irrigation supply and plant tissue samples. The measurement unit is connected to the irrigation supply main line 405 using a sample line 406 where the irrigation supply can be connected to port for the measurement unit. In this case, the measurement unit 401 is most likely to be located at or near an irrigation pumping or fertigation system 403. Fertigation is the delivery of fertilizer solutions to the irrigation water supply so that it is delivered with the irrigation events to the field. Fertigation stations or systems are generally comprised of one or more chemical tanks with either a pump or inductor chemical feed system and valve 403 to draw the fertilizer solution into the irrigation supply line. Often, fertigation stations are co-located with irrigation pump stations 404 where an irrigation supply pump delivers irrigation water to an irrigation supply line, such as irrigation supply main pipeline 405.

The field sampling device is configured to periodically collect, and optionally monitor or analyze, the one or more types of samples to determine the content of at least one nutrient or key environmental quality parameter in the one or more types of samples. For instance, in a typical agricultural context, soil moisture varies on an hourly basis. Appropriate collection intervals in that context are on the order of minutes. For instance, a fifteen minute interval may provide enough granularity to recognize variations in the soil moisture level, and a five minute interval provides three times that. Other variables may have a rate of change measured on the order of days, in which case hourly intervals between analyses of different collections can be sufficient. Preferably, the interval between a first analysis and the next analysis is smaller than the rate of change of the variable being analyzed by enough of a margin so that, as the analyses continue over time, non-negligible changes in the variable (e.g., nutrient content, etc.) can be identified. Those of ordinary skill in the art will readily recognize those changes that are non-negligible for a particular variable in a particular setting. Collection or analysis of samples in this fashion is referred to herein as occurring in “real-time.”

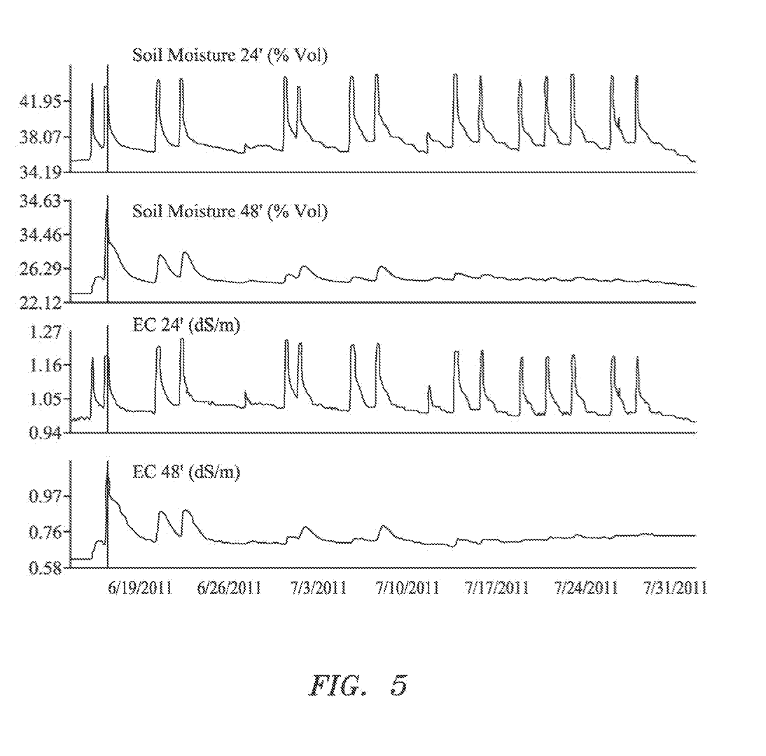

In some embodiments, although not limited to such, the intervals may be between about 5 minutes and 4 hours. In one embodiment, the interval is about every 15 minutes. Sampling every 15 minutes provides for a reasonable and realistic measure of change in the soil environment and with irrigation operations. As shown in FIG. 5, soil moisture and electroconductivity (Ec) changes in the soil profile as measured by sensors installed in situ (i.e., directly into the soil matrix) can reasonably detect and determine changes in both moisture and Ec over time. The chart shown in FIG. 5 illustrates the changes measured in hourly increments by averaging 15-minute data captured within each hour. Changes in nutrient and environmental conditions can be profiled over time with sufficient granularity as to enable wetting events and dry intervals to be discerned or identified.

Some advantages of real-time collection are shown with reference to FIG. 5. Four charts are presented in FIG. 5 illustrating soil moisture and electroconductivity (Ec) changes in a soil profile measured in situ at 24 inches and 48 inches, respectively. In all four charts, the X-axis represents a time period of about 6 weeks, typically during summer growing months, such as for example from mid-June to early August, with each labeled interval representing one week or seven days. The X-axis labels are as follows: 6/19/2011, 6/26/2011, 7/3/2011, 7/10/2011, 7/17/2011, 7/24/2011 and 7/31/2011. Data was collected at 15 minute intervals. The first, uppermost chart shows soil moisture at a depth of 24″ in units of % Vol. The Y-axis labels are 34.19, 38.07 and 41.95. The second chart shows soil moisture at a depth of 48″ in units of % Vol. The Y-axis labels are 22.12, 26.29, 30.46 and 34.64. The third chart shows Ec at 24″ units of dS/m. The Y-axis labels are 0.94, 1.05, 1.16 and 1.27. The fourth, lowermost chart shows Ec at 48″ in units of dS/m. The Y-axis labels are 0.59, 0.78 and 0.97.

The prior art measurement techniques collect a single sample at a point in space and time. These techniques are limited in that they only allow growers to profile an area of their farm so that they can determine the total nutrient load required to be applied to the field over the crop production cycle, and do not provide a real-time, or accurate, nutrient requirement of the field. Further, the general guidelines used by growers today are based on large safety factors of uncertainty to ensure that sufficient nutrients are available to the crop during its maturation process. This uncertainty has led to over-fertilization being generally practiced by growers. Real-time measures for nutrient and environmental conditions in soil pore water can be performed based on the subject matter disclosed herein, and thus growers will have direct knowledge of the available nutrient load for their crops and be able to adjust their application rates to meet crop demands directly rather than using excess nutrient loads to ensure sufficient quantities are available over the crop production season.

While one illustrative embodiment is described using an interval of about 15-minute increments for data capture (and this interval may be selected as a default time interval for the sampling unit) the system can be operated across a range of time intervals for sampling measures. When using the device based on current ISE technology, the smallest time interval practical for this device is about 5 minutes due to the need to stabilize ISE measures and perform the necessary fluid flows to transfer water samples between sampling chambers and rinse solutions. In some embodiments, the high range or largest time interval practice for profiling a wetting event for nutrient load and transport is about 4 hours. Other time intervals for the high range of the interval are suitable, however about 4 hours is a pragmatic upper bound. The maximum duration of irrigation wetting events range from 48 to 72 hours. In order to profile the changes in nutrient load through the irrigation wetting event, a minimum of six (6) and preferably ten (10) or more sampling results are preferred to properly characterize the nutrient levels and their changes through the wetting event. Thus a pragmatic limit of 4 hours between sampling events is reasonable, however other intervals may be used as taught herein, and the disclosure is not to be limited to the specific examples described. The operational time interval for measurements taken from the soil pore water sampling operation can be set as a 15-minute interval by default and can be adjusted by the user.

The sampling unit is particularly suited for installation in agricultural fields as either a permanent or transient installation. In the case where the crop type is a permanent crop such as is the case of orchards and vineyards, the sampling unit is intended to be installed and left resident in the field throughout the life of the unit. Where the crop types are annual or seasonal in their production, the sampling unit can be installed post-planting and removed pre-harvest. The installation of the soil pore water sampling unit is performed with minimum disturbance to the soil environment, much like the installation methods used today for placing sensors in situ in soil environments. After a couple of weeks following installation of the system, the sampling chambers act similarly to other sensors that are installed directly into the soil. Therefore, the soil pore wafer collection unit produces samples that are representative of the in situ conditions of the ambient soils. This differs substantially from other methods of sampling for nutrient or environmental conditions in ambient soils.

Some embodiments capture nutrient and environmental quality measurements from a multiplicity of media or sources important to agricultural field operations. In prior art techniques, samples are performed discretely using field or laboratory methods to determine the nutrient content of soils and plant tissue. No sampling is typically practiced to determine the nutrient or wafer quality conditions for irrigation supplies. The sampling described here enables real-time measurements of both soil pore water and irrigation supplies for nutrients and environmental quality conditions. It also includes the ability to have episodic (or grab) samples of plant tissue processed directly in the field by the user. Therefore, the system performs measurements using Ion Selective Electrodes (ISEs) in the following media: ambient soil pore water; irrigation water supply; and plant tissue.

Plant tissue sampling allows a grower to validate the nutrient state of its crop while assessing the ambient nutrient load available in the soil environment for the crop. The sampling of the irrigation supply water provides the data preferred for determining the input nutrient load and/or background water quality conditions. The ambient soil pore water data indicates the availability of nutrients available at any instantaneous point in time in the root zone environment of the soils. Measurement of all three media provides the data needed to assess and validate nutrient load, availability and uptake by the crop.

The capture of soil pore water or irrigation supply measures in real-time can be initiated by triggers. The sampling unit collects data from other external sensors placed on the irrigation supply line and within the soil environment, namely, soil moisture. Pressure sensors are used to detect changes in irrigation supply operation in the field. The pressure sensor is connected to the sampling unit and when flow is detected in the irrigation supply, the system's microprocessor triggers a sampling event for the irrigation supply and initiates procedures to prepare the system to begin soil pore sampling. The soil moisture sensor is installed so that infiltration of irrigation supplies into the soil environment can be detected. The sampling unit's microprocessor is programmed to initiate the soil pore water sampling routine once the threshold trigger for soil moisture from the installed soil moisture sensor is reached. The soil moisture threshold for triggering a sampling event is set by the user after initial wetting tests are performed by the installation team.

Current methods of using ISE sensors in agricultural settings are focused on direct installation of the sensors within the soil environment. This method of deploying ISE sensors has not been proven successful when left in place for extended periods of time (e.g., weeks). The amount of sensor fouling and loss of calibration have resulted in poor data quality. Therefore, beyond research initiatives where sensors are installed and uninstalled frequently, there has been no successful deployment of ISEs directly into soil environments.

To overcome this known difficulty, the deployment of ISE technology in field environments is performed in such a manner as to ensure the long-term operational stability of the sensors while in the field without the need for frequent maintenance. Several features that permit the long term stability of ISE use for real-time field measurements of nutrient and environmental conditions include: housing for the ISEs is in a weather-proofed (NEMA-4) enclosure; the sensor tips of the ISEs are continuously submerged in either sample water streams, deionized (DI) water, cleaning solvent(s), or calibration standard solutions; the microprocessor of the sampling unit manages the fluid stream operation of the sampling unit so that the sensor “health” can be determined as part of the data acquisition process and maintenance can be scheduled; the housing is hinged bar opening in order to access components of the sampling system; and the ISEs are delivered to the unit in cartridge assemblies so that they can be easily removed for maintenance and/or replacement.

With these features, the performance of ISEs can be maximized and managed with only a limited amount of field maintenance and support.

One example method is illustrated in the flowchart shown in FIG. 6. In general, collection of field samples from an agricultural environment is provided at step 600. The field samples may be obtained from a variety of sources, specifically any one or more of: soil pore water 602, the irrigation supply 604, and the crop canopy or fruit 606. In this embodiment, soil pore water collection is initiated based on defined event trigger conditions, for example as automated based on local irrigation sensors, or based on manual/interval based trigger conditions, step 608. Irrigation supply sampling can be initiated by any one of: (1) sensors that monitor the irrigation system operation that are linked (such as in communication with the field sampler device) or directly connected to the device; or (2) by user-initiated events (i.e. manual operation) at step 610. Crop canopy or fruit samples are initiated by the user as show in step 612.

Once samples are collected, detection of ions or anions of interest are detected using ISE sensors as shown in step 614. The raw data from the ISE sensors is then transformed to human usable data at step 616.

The transformed data is processed for transmission to one or more remote data servers at step 618. The data is transmitted to the one or more remote data servers at step 620. Data is stored in the event of transmission failure and then resubmitted once connections are available, as shown in steps 622 and 624, respectively.

The field sampling device 100 is controlled and operated by one or more microprocessor units 130 which is preferably, but not necessarily, housed in the measurement unit 104. In some embodiments the microprocessor unit 130 is programmed to operate the unit in the manner shown in the flowchart illustrated in FIG. 7, and described further below.

In general, the microprocessor 130 is configured to carry out sample initiation at step 700, perform ISE sample measurement at step 710, process the ISE measurement at step 720, perform data acquisition and transmission at step 730 and end the monitoring session at step 740.

In the absence of having a sampling event be triggered, the DI Reservoir pump will operate on a defined schedule to circulate DI water from DI reservoir supply in and through the sample reservoir. Sensor signals are captured and transmitted at the frequency defined by the user for this quiescent period with such measurements to be used as an indicator of sensor operational performance.

A sampling event may be initiated under a variety of conditions, referred to as “trigger conditions.” In some embodiments, trigger conditions which initiate a sampling event are defined as: (1) on the basis of a sensor signal or with a user over-ride that a wetting event is underway; or (2) the user manually initiates the sampling events 701. A wetting event is typically the act of irrigating, either artificially or naturally (e.g., rainfall). When an event is signaled, the microprocessor continues to run the DI fluid through the manifold 116 and sampling line 118 (see FIG. 1) for a defined time period to fully flush the sample line of any residue from previous sampling events 702.

The following sequence of steps or behaviors is then performed: valve from sampling header to the sampling reservoir is closed; all of the sampling line valves are opened; and the sampling pump is turned on and a low vacuum is pulled across all sampling chambers. This operation continues for throughout the sampling event (sampling pumps on). In some embodiments, sensors will be used to detect fluid fill levels in the collection chambers (108) to ensure positive flow into the sampling chamber. Nearby soil moisture sensors will also be provided in a separate device that can deliver its data or signal to the sampling unit via the wireless telemetry unit where two-way communication with the remote servers or with the LAN enables data to be shared among common devices to validate depth of wetting event penetration and set operational limits on the collection chambers to be actively sampled in any sampling event.

After the low vacuum is pulled across, the sampling line valves are all closed except one; the valve from the sampling header to the sampler reservoir is opened (703); water from the collection chamber associated with the open sampling line is pumped from the collection chamber to the sampling reservoir (704); meanwhile, the microprocessor will have powered on the ISE sensors, allowing them to “warm up” for a sampling event (711); water is circulated through the sampling reservoir until measures from the ISE sensors are captured by the microprocessor (712, 713); the sampling line valves are then sequenced through open/close cycles as samples are processed for each collection chamber, collecting measurements and repeating the sampling process throughout the sampling event period (714); and water from the sampling reservoir is returned to the collection chamber from which it originated. So each of the sample lines has two microtubes for each direction of fluid flow.

This sampling operation is continued until either the wetting event stops based on the sensor signal delivered to the microprocessor, or the soil moisture levels as measured by the representative sensors fall below a defined threshold for sampling (741).

The operational health of the unit is monitored and transmitted as well as the sampling data from the unit to the data acquisition system associated with the unit. The operational health includes measures and assessments such as, but not limited to, one or more of: power supply, sensor responsiveness, sensor drift, sensor outlier values, vacuum pump energy, vacuum pump operational status, vacuum pump hours from last maintenance and total hours, solute reservoir volume, and solute reservoir refill flags.

Multiple solute reservoirs are available to deliver calibration and ISE sensor tip cleaning solutions in addition to DI water. These solutions are used when the diagnostics of ISE sensor health determine that a sensor is operating outside of its performance specifications as set by the user. Tip cleaning and recalibration is performed in an automated manner, controlled by the microprocessor and associated firmware.

All data from the unit is initially transformed into human readable data (721) for local display as well as for preparing data packets for transmission via any suitable means, such as standard wireless or wired communication units (including necessarily a modem), or by a data acquisition system such as a programmable logic controller or some other data acquisition device.

Of particular advantage, one or more variables are user configurable. For example, users can configure the device 100 using available firmware to set preferences for criteria such as but not limited to: sampling frequency, ISE sensors and their configuration, criteria for initiating and stopping sampling events, and remote alarm conditions for user notifications sent via the related communication or data acquisition service, and the like.

The operation of the measurement device incorporates data acquisition and communications of the ISE results to an internet-hosted server system where the data can be processed and used by related software applications (730). Data collection is date/time stamped (731) using the real-time clock available in the microprocessor unit (130) and is then parsed into the proper structure for data delivery via the communication unit (734), which may involve a step of capturing the processed data (733). The communication unit itself operates in a sleep/wake mode and must be returned to a power-on state to establish a communication session (732). Data is then, received, packetized and delivered (735) to the remote servers and its deliver is confirmed (736, 737). In the case where delivery cannot be confirmed, the data packet is stored (738) locally on the microprocessor unit (130) with delivery attempted during the next communication session until confirmation is received by the server.

Upon receipt of a trigger to end the sampling session (740) via sensor signal, user input, manual control, time limitations or other means, sampling line valves (122) will be closed and DI solution will be pumped to the sample channel to rinse the ISE units (741 ). Other solutes may be directed to the sample channel as needed for sensor diagnostics, calibration, and cleaning. The system will be returned to its non-sampling operating routine following the end of the sampling session (742).

FIG. 7 is a flowchart illustrating various operational states of the sampling device in more detail. The sampling device can be operated in up to four operational states, referred to as 1) Deionized Water Sampling; 2) Sampling Event; 3) Calibration Event; and 4) Cleaning Event.

While the various ion selective electrodes (ISEs) are operating in “good health,” e.g., producing acceptable quality data, the sampling event operational mode is triggered by the response of sensors installed into the irrigation system and the ambient soil profile (see FIG. 7). Once a sampling event trigger is exceeded (701), the sample chamber is rinsed using deionized water as needed (702), and then the valves for the sampling chambers are opened (703). Micropumps are used to extract soil pore water from the sample collection lines. When samples from the irrigation line are to be processed, the irrigation system runs under pressure and so sample can be delivered directly to the sampling reservoir.

There is a general procedure used for all sampling operations once the correct solution is delivered to the sampling reservoir: the ISE sample measurement is collected (710) by powering up the probe (711) and collecting readings until a stable reading is captured; the resulting ISE reading is then processed, which is the conversion of the ISE reading into, e.g., the appropriate engineering units when the unit is in a sampling event (720) or deionized water sampling operation; once the data is transformed, it is then further processed and packaged for data delivery using wireless data transmission (730); and the sampling operation continues and the steps are repeated until either the end of the monitoring session (740) is triggered and either deionized water sampling is performed or else another type of operation is required.

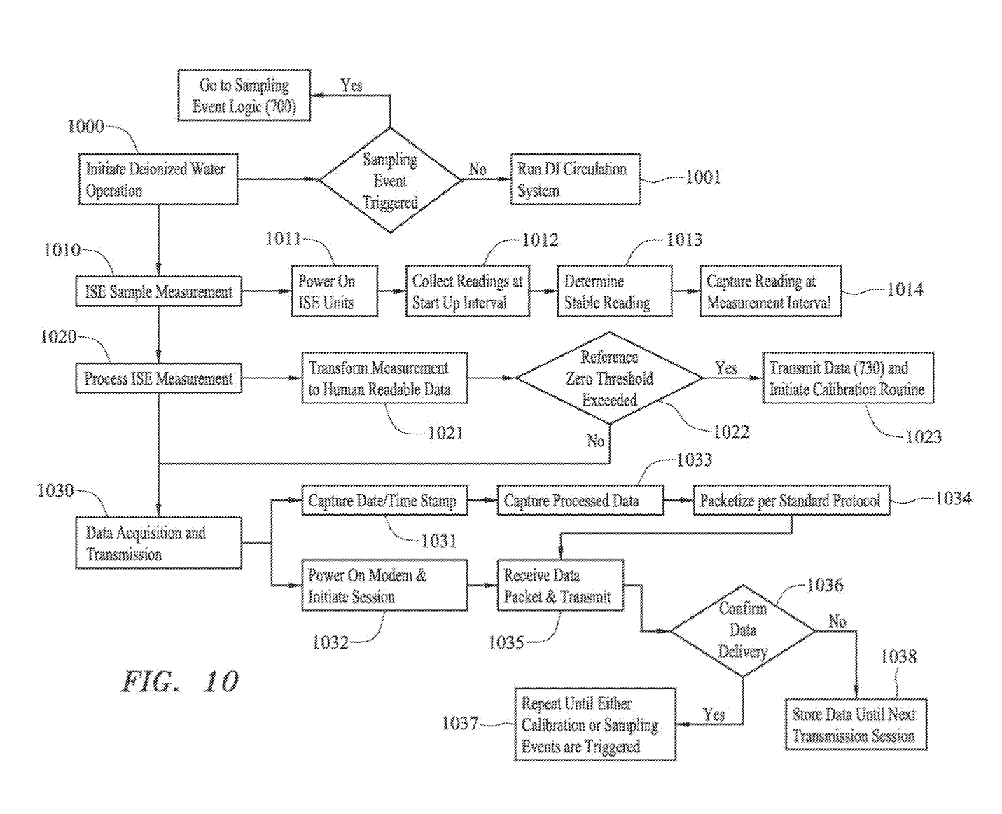

Each of the states is now described in more detail. The deionized water sampling condition is the default state of the sampling device until a trigger condition is met for the device to enter an “event” state (e.g., sampling, calibration, or cleaning). The operation of the sampling device while deionized water is recycled through the sampling reservoir is described in FIG. 10. The sample key components of device operation as that required for sampling events are found with the exception of the logic to control the operation of valves and pumps for various sampling collection chambers. Once a stable reading is captured by the device's microprocessor, a check is performed to test whether the reference zero trigger is exceeded at step 1022. If the reference zero trigger is exceeded, then the sensor calibration event is initiated if the sensor(s) do not have a cleaning routine that is to be performed.

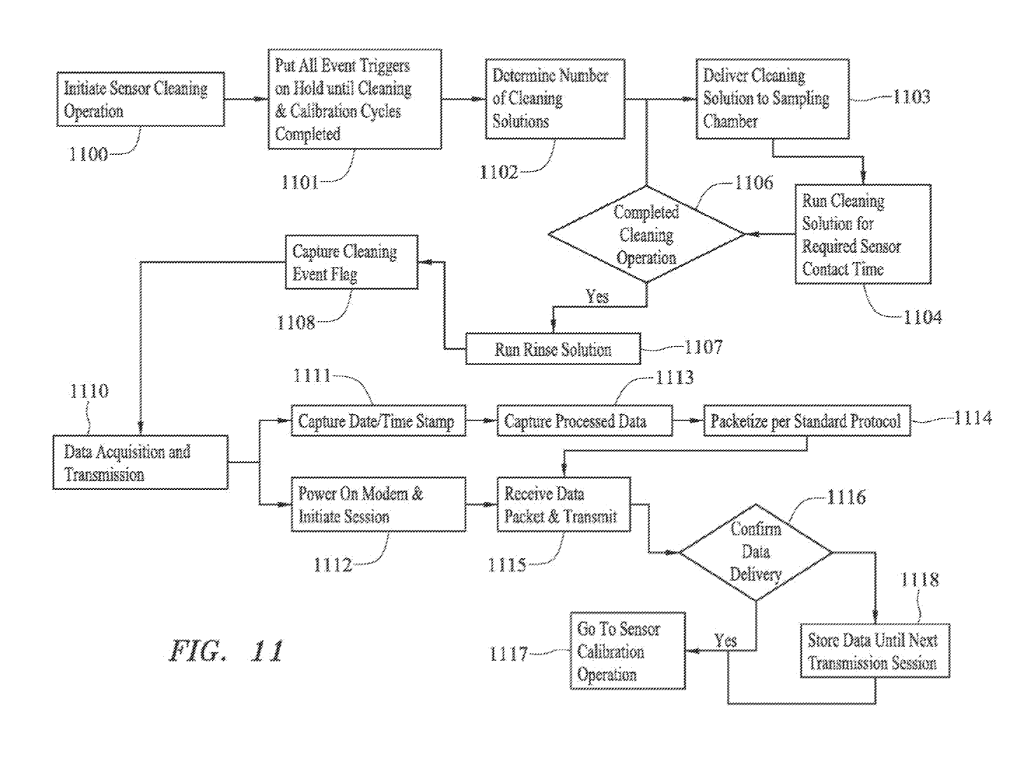

Cleaning routines are performed when an ISE's long term operation will benefit from the use of such solutions. All liquid used from the cleaning solution reservoir are recycled to that reservoir for future use. The logic for the sensor cleaning operation is presented in FIG. 11. No sensor measurements are captured during the cleaning cycle, and in fact, all sensors are powered down during cleaning operations. Depending on the array of ISEs in a specific sampling device, multiple cleaning solutions may be needed. The specific device is configured with the number of cleaning solutions to process during cleaning events (1102). Cleaning solutions are then delivered to the sampling reservoir and pumped for a defined minimum time in order to effectuate sensor cleaning (1104). Once all cleaning solutions have been processed, the sampling device microprocessor creates a cleaning event flag (1108) which is transmitted using the standard data acquisition and transmission procedures to the remote servers (1110). At the completion of all cleaning operations and final sample reservoir rinse with deionized water (1107), the unit will then automatically be placed into the calibration event mode (1117).

During the calibration event, all routine sampling or deionized water operations are prevented from taking place until the calibration event is completed. The calibration method to be used (and therefore, the number of calibration solutions to be processed) is defined at the time that the device is deployed in the field (1202). Each calibration solution is delivered to the sampling reservoir (1203) and sample readings are captured after the sensor has achieved a stable reading (1213). The ISE data processing includes storing all sensor readings until all calibration solutions have been processed. The data is stored (1221) on the local microprocessor, and then used to create a new best-fit regression curve (1222) using the sensor-appropriate function to transform sensor readings to human readable engineering units. The resulting calibration curve is stored on the microprocessor and is used to transform future sensor readings (1223). Flags and the data results from the calibration event are organized and delivered wirelessly to remote servers (1230). A final rinse cycle is performed (1237) before returning the device to its current sampling state (either deionized water or event sampling) (1239).

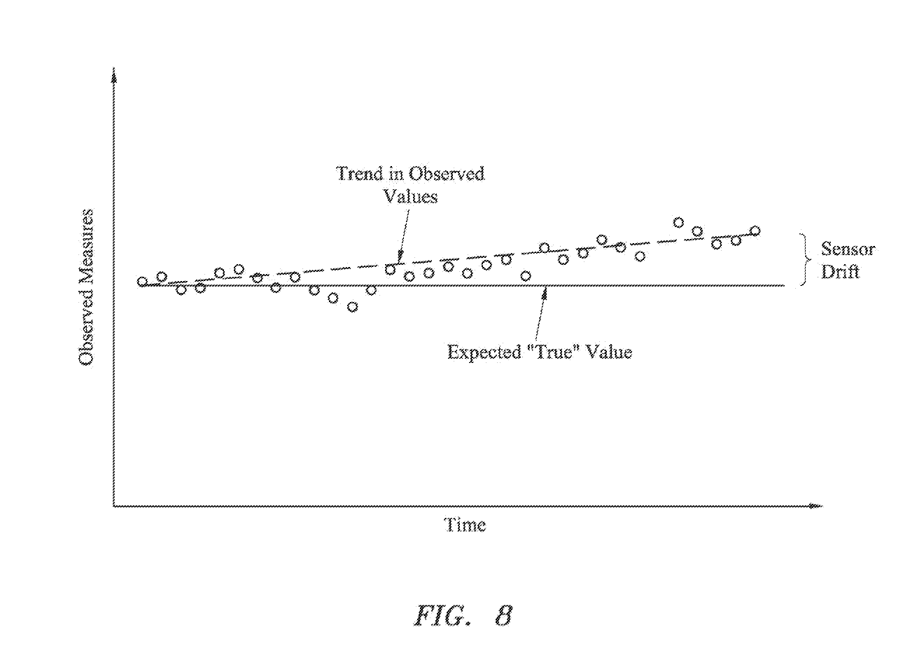

One of the important challenges to providing field-deployed, real-time monitoring for nutrients and other environmental quality indicators in agricultural settings is the ability to maintain the performance of the ion selective electrodes (ISEs) to produce quality data over extended periods of time (e.g., multiple mouths). ISE data quality can degrade over time due to many reasons: fouling of the sensor tips, loss of detection sensitivity (especially if the sensor tips are allowed to dry out over long periods), and electronic noise or disturbances in the sensor. The most common effect of these degradations in sensor performance is known as “drift” where the ability of the sensor to return the same measurement for a given concentration of an analyte “drifts” from the true measurement to something other than the true concentration as illustrated in FIG. 8.

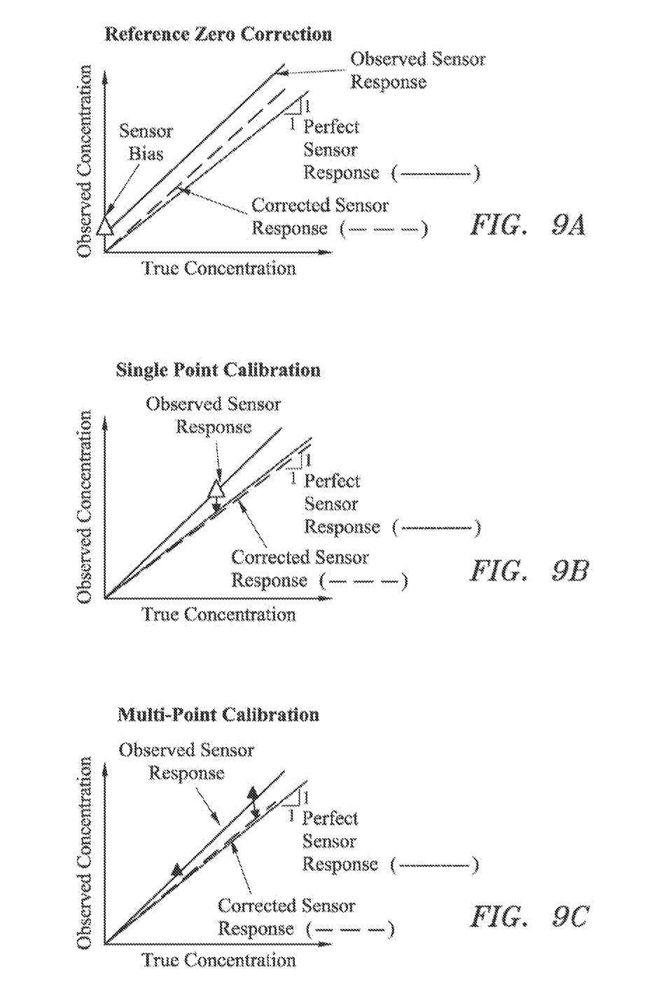

Sensor drift can result in a consistent bias across a measurement range—a positive or negative offset in the measurement outcome. This type of error can be detected when multiple measurements are collected from samples of known concentration. Samples of known concentration are provided by using calibration standard solutions. To correct for measurement error caused by sensor drift, a combination of two procedures is needed. FIGS. 9A-C show three example embodiments of methods for correcting sensor drift or performance. FIG. 9A illustrates sensor correction using what is referred to herein as reference zero correction. According to this embodiment, deionized water is used, which by definition, contains negligible concentrations of ions (effectively zero levels). Nutrients and environmental quality indicators are commonly expressed in solution as ions. The absence of ions means that the sensor measurement response when exposed to deionized solutions should be zero. As part of the routine operation of the sampler system, the ISEs will routinely be exposed to deionized water and measurements will be collected over time. A threshold offset of the measurement response will be used as a trigger for a complete sensor calibration event. The measurement offset from zero as determined by comparing the sensor response to the “true” zero while taking measurements while circulating deionized water represents a sensor “bias” as indicated in FIG. 9A. This bias will be corrected from those sampling event measurements between calibration events.

Sensor correction can also be achieved by standard sensor calibration methods, such as what is referred to herein as “single point calibration” (shown in FIG. 9B) or what is referred to herein as “multi-point calibration” (shown in FIG. 9C). Sensor measurements provide an electrical response proportional to the concentration of the analyte. The transformation of the sensor signal to engineering units (e.g., concentration) can be mathematically described, such as by linear or non-linear best fit curves of analyte concentration versus electrical signal. This best fit curve serves as a calibration curve. Correction of sensor drift occurs through re-calibration of the sensor after re-zeroing.

Calibration typically requires that the sensor capture measurements from standard solutions having a known (“true”) concentration. With single point calibration, the zero reference response and the sensor response from the calibration standard are used to best fit a sensor response versus electrical response. In this case, typically a linear best fit curve is provided for the sensor. A more robust calibration approach uses multi-point calibration where multiple standard solutions are used along with the reference zero response to best fit a new curve for sensor responses in measurement units and electrical response. A best fit sensor response curve between the values of zero and the calibration response measures is determined so that the offset between the true values of the standards and the response values can be determined. Corrections can be made to subsequent measures to represent a calibrated reading per the correction shown above.

According to some embodiments, methods of sensor correction for measurement errors are provided. Sensor data from the field ISE sensors must be transformed from “raw” electrical signals into human readable engineering units. The relationship between sensor electrical signals and human readable engineering units is the calibration equation and can take any of a number of mathematical forms, but the most common is a linear response:

C=a*(mV)+b

Where “C” is the concentration of the analyte being measured by the ISE, “mV” is the electrical signal returned from the ISE, and both “a” and “b” are the coefficients resulting from a best-fit analysis of the sensor response versus known concentration data. Manufacturers provide an initial calibration equation for the transformation of the data signals from their sensors. The calibration provided by the manufacturer defines the mathematical form of the calibration to be used for future re-calibrations.

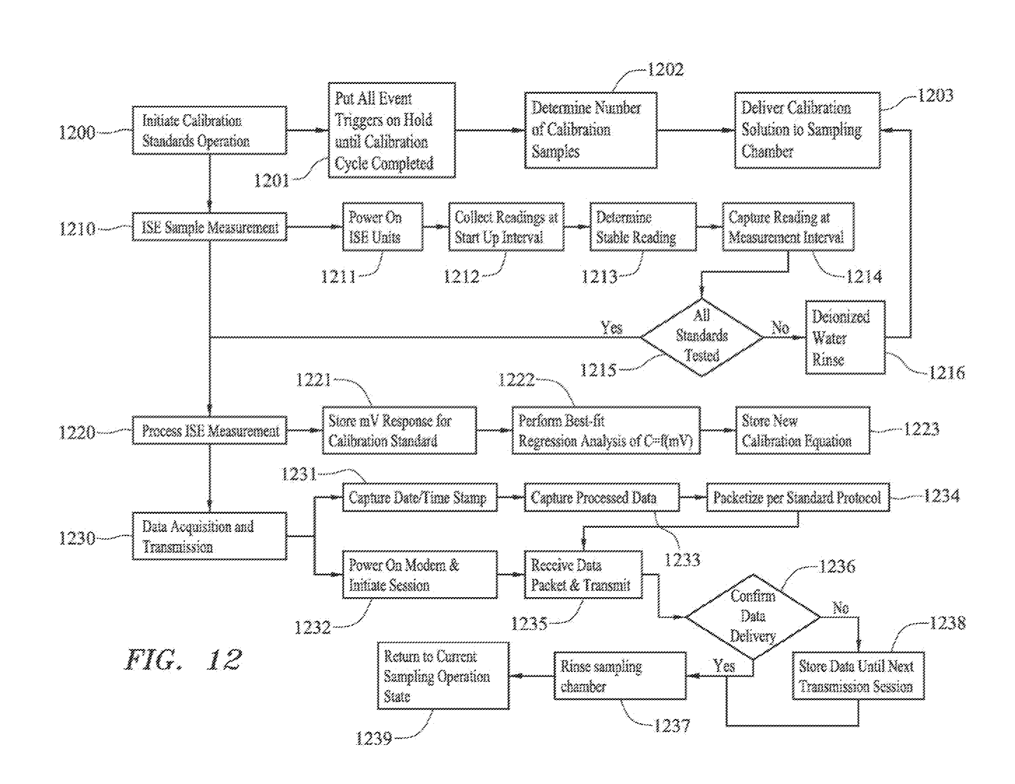

Based on the triggers set to initiate a calibration event, the field monitoring device is entered into a Calibration operational state and cannot return to any other operational state until the calibration routine is completed as illustrated with reference to FIG. 12. Based on the type of ISE, the number of required calibration standard solutions is identified (1202). The device then delivers the first calibration standard of known concentration to the sampling chamber. The sampling device powers up the ISE (1211) and starts collecting readings once the calibration event is triggered (1212). Once a stable reading is obtained from the ISE (1213), the sensor response (mV) is stored, a deionized water rinse is initiated by the device (1216), and then subsequent calibration standards are delivered to the sampling chamber. This process is repeated until all calibration standards have been processed by the device and mV readings are available for all standard concentrations.

Since the initiation of the calibration event is based on the sensor response to the reference zero routine, calibration curve fitting routines use the last reference zero sensor response to assure that the calibration curve extends for the full scale. The microprocessor for the device stores each of the mV responses for all calibration standards and the most recent reference zero reading (1221). Based on the specific ISE, the form of the calibration equation is determined. Standard statistical methods are used to determine the best-fit curve for the calibration data (1222). The new calibration equation is then stored in the microprocessor (1223) for use on future sensor readings until the next calibration event.

The data for the calibration event is processed for transmission (1230), packetized and delivered wirelessly (1235) to hosted servers that are located at a remove from the field. If the data delivery cannot be confirmed (1236), then the calibration data packet is stored and transmission is attempted during the next sample transmission session (1238). When data transmission is completed or once the non-transmitted data is stored, the sampling chamber is rinsed using deionized water and the sampling device is returned to sampling state resulting from the most recent conditions for the various triggers.

The operation of the measurement device can include data acquisition and communications of the ISE results to an internet-hosted server system where the data can be processed and used by related software applications.

Certain embodiments described herein provide for an operational schema for field sampling of soil pore water and use of ISE sensors in a manner that enables the ISE sensors to remain stable and accurate over long periods of time.

Unit 100 is fully deliverable to the field monitoring environment and can be deployed in such a manner that reliable data delivery to a remote data server is feasible regardless of terrain or crop canopy.

In summary, embodiments described herein include many valuable and varying features and advantages, such as but not limited to, one of more of the following:

An operational schema for field sampling of soil pore water and use of ISE sensors in a manner that enables the ISE sensors to remain stable and accurate over long periods of time unlike the prior art systems;

An operational logic and system defined to trigger sampling events based on the wetted condition of the local soils;

A device that enables easy maintenance by compartmental arrangement of components for access by maintenance service personnel; and,

The integration of sensor “meter” to interpret sensor signals with data packetization for transmitting data to third party devices (modems or data acquisition units) using a plurality of standard data communication protocols.

As set forth above, the systems and devices described herein are suitable for operation to sample multiple sources for ion content, including direct measurement of the irrigation supply (with and without fertilizer addition) as well as multiple depths within the soil profile, depending on the effective root zone of the crop. The sample collection unit has multiple chambers at pre-selected depths to profile the soil pore water conditions representative of those depths. The sampling assembly with manifold and micropump systems can be used to exert vacuum pressures so that soil pore water flow during irrigation events is induced to collect in the collection chambers and then transferred to the sampling chamber using the programmed operational logic for the system. Intermittent flushing of the sampling chamber with a neutral water supply (e.g., deionized wafer, and the like) occurs between representative samples so that there is only negligible opportunity for cross-contamination of a given sample from previously sampled sources.

In additional embodiment, the sample processing unit can be mounted on the measurement unit for processing and generating appropriate samples from plant tissue. This would provide the grower with the opportunity to measure not only their applied nutrients in their irrigation supply, and the available nutrients in the soil pore water environment, but also the plant nutrient content periodically as a check measure on plant uptake and nutrient fate.

In addition, the operation of this system incorporates data acquisition and communication of the ISE detection results to an internet-hosted server system where the data can be processed and used by related software applications.

Of particular advantage, with the ability to deliver growers with time series data on nutrient concentrations in the available soil moisture delivered to their crops, a more informed decision-support system can be created to support a “just in time” approach to nutrient management.

Certain multi-step operational flows are described herein, for instance, with respect to FIGS. 6, 7, and 10-12. Each of these steps, performed individually, as a whole, or in combinations described and implied but not explicitly shown, bear a relationship to promoting the growth of the agricultural crop. In other words, through the many mechanisms described, these steps transform the crop (an article) from a first state to a second (healthier, or more robust, or more mature) state or, viewed differently, maintain the crop from transforming from a first state to a second (less healthy or less robust) state. It is intended that claims covering the performance of any of these steps in the context of transforming the crop from a first state to a second state be within the scope of this disclosure.

It should be recognized that a number of variations of the above-identified embodiments will be obvious to one of ordinary skill in the art in view of the foregoing description and teaching. Accordingly, the inventive subject matter is not to be limited by those specific embodiments of the systems, devices, and methods described herein.

1. A device-based system adapted for operation in an agricultural setting, comprising:
a first unit deployable in soil and adapted to collect a soil pore water sample therefrom; and
a second unit coupled to the first unit and adapted to independently monitor for a condition and trigger the collection of the soil pore water sample in the first unit based on the occurrence of the condition.

2. The system of claim 1, wherein the second unit is adapted to analyze the soil pore water sample to determine the content of a nutrient in the soil pore water sample or an environmental quality parameter of the soil pore water sample.

3. The system of claim 2, wherein the nutrient is nitrogen, phosphorous, potassium, or boron.

4. The system of claim 2, wherein the environmental quality parameter is calcium content, magnesium content, carbonate content, sulfur compound content, pH, electroconductivity, or temperature.

5. The system of claim 2, wherein the first unit further comprises an elongate tube having a collection chamber formed therein.

6. The system of claim 5, wherein the first unit comprises a plurality of elongate tubes, each having a collection chamber located therein, wherein each of the collection chambers is positioned such that they are present at different depths when the first unit is deployed in the soil.

7. The system of claim 5, wherein the collection chamber includes a wall, the wall having an opening to allow soil pore water to flow in.

8. The system of claim 5, wherein the collection chamber includes a porous region to allow soil pore water to flow in.

9. The system of claim 5, further comprising:
a sampling assembly comprising:
a manifold;
a pump coupled to the manifold;
a sampling reservoir; and
a sampling line coupled to the manifold and having an open distal end, wherein the open distal end is in communication with the collection chamber to draw the soil pore water sample into the collection chamber.

10. The system of claim 9, wherein the sampling assembly further comprises a port coupled with the manifold, the port adapted to allow an irrigation supply sample to pass into the manifold.

11. The system of claim 2, wherein the second unit further comprises an ion selective electrode (ISE) sensor, the ISE sensor adapted to detect ions or anions representative of the content of the nutrient in the soil pore water sample.

12. The system of claim 2, wherein the second unit further comprises a microprocessor configured to process data representative of the content of the nutrient in the soil pore water sample or data representative of the environmental quality parameter of the soil pore water sample.

13. The system of claim 1, further comprising a third unit adapted to analyze a plant tissue sample.

14. The system of claim 1, further comprising a data acquisition and communications unit communicatively coupled to the measurement unit and configured to transmit data corresponding to the content of the nutrient to a data server remote from the agricultural field.

15. The system of claim 1, further comprising a solar panel adapted to provide power to the second unit.

16. The system of claim 1, wherein the system is adapted to collect an irrigation water sample, and the second unit is adapted to measure the irrigation water sample.

17. The system of claim 1, wherein the first unit is adapted to periodically collect soil pore water samples.

18. The system of claim 17, wherein the periodic collection takes place at an interval between about 5 minutes and about 4 hours.

19. The system of claim 18, wherein the interval is about 15 minutes.

20. The system of claim 1, wherein the first unit is adapted to periodically collect soil pore water samples without being removed from the soil.

21-62. (canceled)
